(19) United States
(12) Patent Application Publication
(10) Pub. No.: US 2020/0069921 A1
(43) Pub. Date: Mar. 5, 2020

(54) DEVICE FOR OCCLUSION ISOLATION AND REMOVAL

(71) Applicant: Q3 MEDICAL DEVICES LIMITED, Dublin (IE)

(72) Inventor: Eric K. MANGIARDI, Charlotte, NC (US)

(21) Appl. No.: 16/117,730

(22) Filed: Aug. 30, 2018

(51) Int. Cl. A61M 25/10; A61M 25/00

(52) U.S. Cl. CPC A61M 25/104; A61M 25/0074; A61M 1/0023; A61M 2025/109; A61M 25/10184

(57) ABSTRACT

A device for occlusion removal from a bodily lumen is described. The device includes a multi-lumen catheter, a manifold that attaches to the catheter, and an aspiration device that inserts through a lumen of the catheter. A method of removing an occlusion from a bodily lumen is also described, as is a kit containing the device.

### FIELD

The present application is related to medical devices and methods for the isolation and removal of occlusions; and more specifically, to an inflatable balloon catheter device and methods of using the same.

### BACKGROUND

Occlusions are a serious medical condition involving an internal blockage, most often of a blood vessel or organ. Thromboses, or vascular occlusions, are caused by the formation of blood clots in blood vessels, and a blockage occurring within a major vein may cause deep vein thrombosis, among other conditions. In addition, urological, gastrointestinal, ductal, and ophthalmic (retinal) occlusions can occur, partially or totally blocking the flow of bodily fluids through those lumens.

Occlusions are generally treated by the removing or diminishing the blockage. Current methods for this procedure in blood vessels, for example, include alternating insertions of a catheter that breaks up the occlusion with the insertions of a catheter that aspirates the debris of the occlusion from the vessel. However, this procedure is performed with blood still flowing through the vessel, potentially allowing fragments of the occlusion to be carried away from the original site, with the risk of those fragments lodging in other locations. Additionally, this procedure requires repetitive insertion of catheters into the site, raising the risk of damage to the walls of the lumen.

Thus, there is a need for a single-insertion device to isolate occlusions in a lumen by stopping the flow of bodily fluids in the region of the occlusion, facilitating the reduction or removal of the occlusion with minimal disruption to the surrounding tissues and preventing debris from being redistributed in the body.

### SUMMARY

A first aspect of the present application relates to a device for occlusion removal comprising a catheter having a proximal end and a distal end, and an aspiration device. The catheter comprises a main lumen having a proximal opening at the proximal end of the catheter and a distal opening at the distal end of the catheter, the main lumen comprising an inner diameter sufficient to accommodate the passage of the aspiration device. The aspiration device comprises a longitudinal lumen, a connection at the proximal end for a suction device, and at least one opening at or near the distal end.

A second aspect of the present application relates to a method for the removal or reduction of an occlusion at a target site in a bodily lumen of a subject in need thereof. The method comprises the steps of: a) establishing an entry portal into a site that provides access to the target site, b) inserting the distal end of the catheter of the device of claim 5 through the entry portal and guiding it to the target site, c) advancing an aspiration device through the main lumen of the catheter and engaging the occlusion at the target site, and d) aspirating the occlusion, or fragments thereof, through the catheter.

A third aspect of the present application relates to a kit comprising a catheter as described herein and at least one aspiration device.

A fourth aspect of the present application relates to a device for occlusion removal comprising a catheter having a proximal end and a distal end, a balloon mounted at or near the distal end of the catheter, a manifold having a proximal end and a distal end, and an aspiration device; wherein the distal end of the manifold attaches to the proximal end of the catheter; the balloon comprising: a flexible impermeable membrane encircling the catheter, the membrane being bounded by a proximal collar attached to the catheter and a distal collar attached to the catheter; the catheter comprising: a main lumen having a proximal opening at the proximal end of the catheter and a distal opening at the distal end of the catheter, the main lumen comprising an inner diameter sufficient to accommodate the passage of the aspiration device, and an inflation lumen having a proximal opening at the proximal end of the catheter and a distal opening near the distal end of the catheter, wherein the distal opening is between the proximal and distal collars of the balloon; the manifold at its proximal end comprising: a main port that is contiguous with the main lumen of the catheter, wherein the main port accommodates the insertion of the aspiration device through the manifold and into the main lumen of the catheter, and an inflation port that is in fluid communication with the inflation lumen of the catheter, wherein the inflation port is attachable to a syringe or reservoir for inflation of the balloon; and the aspiration device comprising: a longitudinal lumen, a connection at the proximal end for a suction device, and at least one opening at or near the distal end.

A fifth aspect of the present application relates to a method for the removal or reduction of an occlusion at a target site in a bodily lumen of a subject in need thereof, comprising the steps of: establishing an entry portal into a site that provides access to the target site, inserting the distal end of a catheter comprising a balloon mounted at or near the distal end as described herein through the entry portal and guiding it to the target site, inflating the balloon to stop the flow of bodily fluids through the lumen at the target site, advancing an aspiration device through the main lumen of the catheter and engaging the occlusion at the target site, and aspirating the occlusion, or fragments thereof, through the catheter. In some embodiments, the method further comprises the step of deflating the balloon. In further embodiments, the method further comprises the step of withdrawing the catheter from the entry portal.

### BRIEF DESCRIPTION OF THE DRAWINGS

The present invention can be better understood by reference to the following drawings. The drawings are merely exemplary to illustrate certain features that may be used singularly or in combination with other features and the present invention should not be limited to the embodiments shown.

FIG. 1 is a perspective view of an exemplary embodiment of a catheter assembly of an occlusion removal device of the present application.

FIG. 2 is a cross-section view of an exemplary embodiment of a catheter of an occlusion removal device of the present application.

FIG. 3 shows exemplary distal catheter tip designs for femoral, radial and brachial cannulation procedures.

FIG. 4 shows exemplary distal catheter tip designs for femoral cannulation procedures.

FIG. 5 is a longitudinal schematic view of the architecture of a catheter of an occlusion removal device of the present application.

FIG. 6 is a perspective view of an exemplary balloon member of a catheter assembly of an occlusion removal device of the present application.

FIG. 7 is a perspective view of an exemplary manifold and strain relief assembly of a catheter assembly of an occlusion removal device of the present application.

### DETAILED DESCRIPTION

The following detailed description is presented to enable any person skilled in the art to make and use the invention. For purposes of explanation, specific nomenclature is set forth to provide a thorough understanding of the present invention. However, it will be apparent to one skilled in the art that these specific details are not required to practice the invention. Descriptions of specific applications are provided only as representative examples. The present invention is not intended to be limited to the embodiments shown, but is to be accorded the widest possible scope consistent with the principles and features disclosed herein.

This description is intended to be read in connection with the accompanying drawings, which are to be considered part of the entire written description of this application. The drawing figures are not necessarily drawn to scale and certain features of the application may be shown exaggerated in scale or in somewhat schematic form in the interest of clarity and conciseness. In the description, relative terms such as “front,” “back,” “up,” “down,” “top,” “bottom,” “upper,” and “lower,” as well as derivatives thereof, should be construed to refer to the orientation as then described or as shown in the drawing figure under discussion. These relative terms are for convenience of description and normally are not intended to require a particular orientation. Terms concerning attachments, coupling and the like, such as “connected,” “mounted,” and “attached,” refer to a relationship where structures are secured or attached to one another either directly or indirectly through intervening structures, as well as both movable or rigid attachments or relationships, unless expressly described otherwise.

Unless otherwise noted, technical terms are used according to conventional usage. However, as used herein, the following definitions may be useful in aiding the skilled practitioner in understanding the invention. Such definitions shall be applied, unless a different definition is given in the claims or elsewhere in this specification.

As used herein, the terms “horizontal” and “vertical,” and derivatives of those terms, are used in respect to their relationship to the plane defined by the lengthwise body of the aspiration device of the present invention. “Horizontal” refers to the plane that can, for example, pass through the lengthwise body of the aspiration device, while “vertical” refers to a plane that is perpendicular to the horizontal plane.

The term “proximal” end of a device, or a part of a device, as used herein, is the end that is towards the practitioner holding or operating the device. The “distal” end of a device, or a part of a device, as used herein, is the end that is farthest from the practitioner or towards the subject into whom the device, or part of the device, is to be delivered.

As used herein, the term “advance,” and derivatives thereof, refers to the movement of the device, or an element thereof, in a proximal-to-distal direction.

As used herein, the term “withdraw,” and derivatives thereof, refers to the movement of the device, or an element thereof, in a distal-to-proximal direction.

As used herein, the term “subject” refers to an animal. In some embodiments, the subject is a mammal. In some embodiments, the subject is a human. In some embodiments, the subject is a pet.

The term “mammal” is intended to encompass a singular “mammal” and plural “mammals,” and includes, but is not limited to humans; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and bears. In some preferred embodiments, a mammal is a human.

The term “bodily lumen” as used herein, refers to any cavity within the body of a subject. In some embodiments, the lumen may be a cavity within a blood vessel. In some embodiments, the lumen may be a cavity within an organ.

The present application relates to a catheter-based device for removing or reducing an occlusion at a target site from a bodily lumen. In some embodiments, the device is an aspiration device. In order to prevent migration of fragments of the occlusion from the target site to other locations in the body, the present device also provides a balloon for blocking the flow of bodily fluids at the target site during the process of removing or reducing the occlusion. The device combines the functions of breaking up the occlusion and aspirating the remains thereof from the target site in a single device, meaning the catheter only needs to be inserted a single time and is left in situ throughout the procedure, thus further minimizing the risk of tissue damage at the site. In addition, the present device is also ideal for obtaining tissue samples from within a lumen, such as for a biopsy of suspected cancerous tissue.

A first aspect of the present application relates to a device for occlusion removal comprising a catheter having a proximal end and a distal end, and an aspiration device. The catheter comprises a main lumen having a proximal opening at the proximal end of the catheter and a distal opening at the distal end of the catheter, the main lumen comprising an inner diameter sufficient to accommodate the passage of the aspiration device. The aspiration device comprises a longitudinal lumen, a connection at the proximal end for a suction device, and at least one opening at or near the distal end.

In some embodiments, the device further comprises a manifold at its proximal end, wherein the manifold comprises a main port that is contiguous with the main lumen of the catheter and accommodates the insertion of the aspiration device through the manifold and into the main lumen of the catheter. In further embodiments, the main port further attaches to a handle.

In some embodiments, the device further comprises a balloon mounted at or near the distal end of the catheter. The balloon comprises a flexible impermeable membrane encircling the catheter, the membrane being bounded by a proximal collar attached to the catheter and a distal collar attached to the catheter; and the catheter further comprises an inflation lumen having a proximal opening at the proximal end of the catheter and a distal opening near the distal end of the catheter, wherein the distal opening is between the proximal and distal collars of the balloon. In further embodiments, the device further comprises a manifold at its proximal end, with the manifold comprising a main port that is contiguous with the main lumen of the catheter, wherein the main port accommodates the insertion of the aspiration device through the manifold and into the main lumen of the catheter, and an inflation port that is in fluid communication with the inflation lumen of the catheter, wherein the inflation port is attachable to a syringe or reservoir for inflation of the balloon.

In some embodiments, the device comprises a radio opaque marker at the distal end of the catheter. In further embodiments, the radio opaque marker is a tantalum marker.

A second aspect of the present application relates to a method for the removal or reduction of an occlusion at a target site in a bodily lumen of a subject in need thereof. The method comprises the steps of: a) establishing an entry portal into a site that provides access to the target site, b) inserting the distal end of the catheter of the device of claim 5 through the entry portal and guiding it to the target site, c) advancing an aspiration device through the main lumen of the catheter and engaging the occlusion at the target site, and d) aspirating the occlusion, or fragments thereof, through the catheter.

In some embodiments, the method further comprising the step of inserting a guide wire through the entry portal prior to step b), wherein the catheter is introduced through the entry portal in step b) by sliding the main lumen of the catheter onto the guidewire.

In some embodiments, engaging the occlusion comprises dislodging it intact for aspiration from the target site.

In other embodiments, engaging the occlusion comprises breaking the occlusion into fragments for aspiration from the target site.

In some embodiments, the occlusion is selected from the group consisting of a thrombus, calcification, lipid and cholesterol.

In some embodiments, the bodily lumen is a blood vessel. In further embodiments, the blood vessel is a coronary artery.

In other embodiments, the bodily lumen is selected from the group consisting of ureter, urethra, bile duct and pancreatic duct.

In some embodiments, the method further comprises the step of withdrawing the catheter from the entry portal.

A third aspect of the present application relates to a kit comprising a catheter as described herein and at least one aspiration device.

In some embodiments, the kit comprises a trocar or Seldinger needle.

A fourth aspect of the present application relates to a device for occlusion removal comprising a catheter having a proximal end and a distal end, a balloon mounted at or near the distal end of the catheter, a manifold having a proximal end and a distal end, and an aspiration device; wherein the distal end of the manifold attaches to the proximal end of the catheter; the balloon comprising: a flexible impermeable membrane encircling the catheter, the membrane being bounded by a proximal collar attached to the catheter and a distal collar attached to the catheter; the catheter comprising: a main lumen having a proximal opening at the proximal end of the catheter and a distal opening at the distal end of the catheter, the main lumen comprising an inner diameter sufficient to accommodate the passage of the aspiration device, and an inflation lumen having a proximal opening at the proximal end of the catheter and a distal opening near the distal end of the catheter, wherein the distal opening is between the proximal and distal collars of the balloon; the manifold at its proximal end comprising: a main port that is contiguous with the main lumen of the catheter, wherein the main port accommodates the insertion of the aspiration device through the manifold and into the main lumen of the catheter, and an inflation port that is in fluid communication with the inflation lumen of the catheter, wherein the inflation port is attachable to a syringe or reservoir for inflation of the balloon; and the aspiration device comprising: a longitudinal lumen, a connection at the proximal end for a suction device, and at least one opening at or near the distal end.

A fifth aspect of the present application relates to a method for the removal or reduction of an occlusion at a target site in a bodily lumen of a subject in need thereof, comprising the steps of: establishing an entry portal into a site that provides access to the target site, inserting the distal end of a catheter comprising a balloon mounted at or near the distal end as described herein through the entry portal and guiding it to the target site, inflating the balloon to stop the flow of bodily fluids through the lumen at the target site, advancing an aspiration device through the main lumen of the catheter and engaging the occlusion at the target site, and aspirating the occlusion, or fragments thereof, through the catheter. In some embodiments, the method further comprises the step of deflating the balloon. In further embodiments, the method further comprises the step of withdrawing the catheter from the entry portal.

In some embodiments, the kit comprises a trocar or Seldinger needle.

In some embodiments, the kit comprises a fiber optic nanoscale camera. In some further embodiments, the kit comprises a fiber optic light source.

### Occlusion Removal Device

### Multi-lumen Balloon Catheter Assembly

One aspect of the present application relates to an occlusion removal device comprising a multi-lumen balloon catheter and an aspiration device for removing or reducing an occlusion in a lumen or vessel.

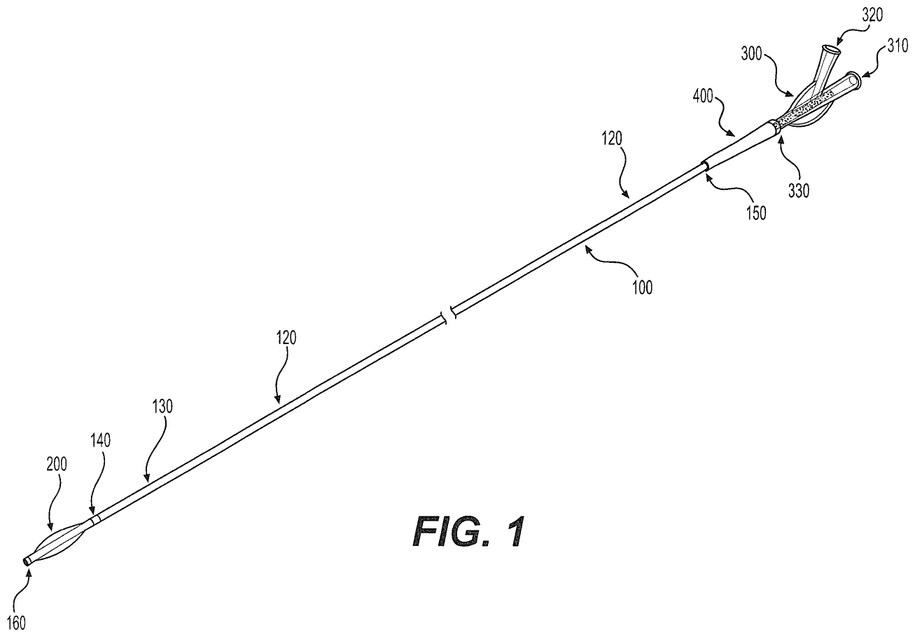

FIG. 1 depicts an exemplary balloon catheter assembly of the present occlusion removal device. The assembly comprises a catheter 100 having at least two longitudinal lumens. The catheter 100 comprises at least a main lumen and an inflation lumen. In some embodiments, the catheter 100 may comprise different zones of stiffness 110, 120, 130, 140, with the zone having the highest stiffness 110 being located most proximally to enhance pushability of the catheter and the zone having the lowest stiffness 140 being located most distally to enhance flexibility through tortuous lumens or vessels. In further embodiments, which may be dependent upon the specific application desired for the occlusion removal device, such as target lumen or entry portal location, the catheter may have 2, 3, 4, 5, 6, 7, 8, 9, 10 or more than 10 zones of stiffness. In other embodiments, the catheter 100 is of a single stiffness throughout its length. In still other embodiments, the stiffness of the catheter 100 decreases gradually or at a constant rate from the proximal end 150 towards the distal end 160.

Still in FIG. 1, a balloon catheter assembly of the present occlusion removal device comprises a balloon 200 at or near the distal end 160 of the catheter 100. The balloon is inflatable with a pharmaceutically acceptable solution, such as, but not limited to water or normal saline. The length of the balloon 200 can be dependent on the specific application desired for the occlusion removal device, such as the target lumen. In some embodiments, the length of the balloon is about 5, 10, 15, 20, 25, 30, 35, 40, 45, 50, 55, 60, 65, 70, 75, 80, 85, 90, 95 or 100 mm long. Likewise, the inflated diameter of the balloon 200 can be dependent on the specific application desired for the occlusion removal device, such as the target lumen. In some embodiments, the inflated diameter of the balloon is about 5, 10, 15, 20, 25, 30, 35, 40, 45, 50, 55, 60, 65, 70, 75, 80, 85, 90, 95, 100, 110 or 120 mm, with the smaller diameters being suited for narrow lumens, such as a blood vessel or duct and the larger diameters being suited for wider lumens, such as an intestine.

Also in FIG. 1, a balloon catheter assembly of the present occlusion removal device comprises a manifold 300, whose distal end 330 attaches, either directly or indirectly, to the proximal end 150 of the catheter 100. In some embodiments, a manifold 300 of the present device comprises a proximal main port 310 that is in fluid communication and aligned with the main lumen of the catheter 100. The main port 310 allows the insertion of an aspiration device into and through the main lumen of the catheter 100. Additionally, after the catheter 100 has been deployed to a target site, the main port 310 allows for the removal of a guide wire from the main lumen and out the proximal end of the assembly. In some embodiments, the main port 310 of the manifold 300 may have an attachment mechanism. The attachment mechanism can be of any suitable form known in the art including, but not limited to threaded, luer slip or luer lock. In some embodiments, the main port 320 can be attached to a handle for the manipulation of the device.

Still in FIG. 1, in some embodiments, a manifold 300 of the present device comprises an inflation port 320 that is in fluid communication with the balloon 200 via the inflation lumen of the catheter. The location of the inflation port 320 on the manifold can be in any location practical for the operation of the device by a practitioner. For example, the inflation port 320 of the manifold 300 may be located on a side of the manifold 300 and generally facing in a proximal direction.

In some embodiments, an area of strain relief 400 is located between the manifold 300 and the catheter 100.

The catheter 100 of the aspiration device may be composed of one or more elongated, monoaxial sections. In some embodiments, sections may have the same diameter, and in other embodiments the diameter may vary between one or more sections. In some embodiments, the sections may have the same stiffness or durometer and in other embodiments the stiffness or durometer, may vary between one or more sections. It is understood that the variations in diameter, stiffness, or durometer may be in a continuous or graduated manner.

In some embodiments, the catheter 100 has a stiffness gradient wherein the stiffness of the catheter 100 is highest at the proximal end and lowest at the distal end. In some embodiments, the stiffness gradient declines continuously from the proximal end to the distal end.

In other embodiments, the stiffness gradient may be constant within a section but vary between sections, with the most proximal section being the most rigid and the most distal section being the most flexible.

In some embodiments, the catheter 100 may comprise a material selected from a nylon, PEBAX (polyether block amide), a polyether block amide that is free of plasticizers, a polyamide, polyetheretherketone (PEEK), any other suitable polymer material, and combinations thereof.

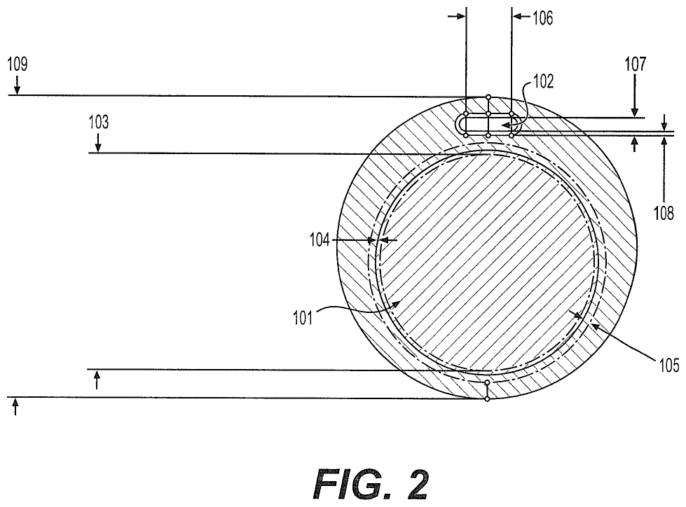

Turning now to FIG. 2, an exemplary cross-section of a catheter 100 having a main lumen 101 and an inflation lumen 102 is depicted. The main lumen 101 is of a sufficient diameter 103 to allow the passage of a guide wire or an aspiration device. The diameter 103 of the main lumen 101 of the catheter 100 can be determined by the application for which the device is intended. For example, a catheter 100 for clearing an obstruction from an intestine will have a main lumen 101 with a greater diameter 103 than a catheter 100 for clearing an obstruction from an aorta, which in turn will have a main lumen 101 with a greater diameter 103 than a catheter 100 for clearing an obstruction from a coronary artery or bile duct. In some embodiments, the main lumen 101 is between about 1 and 10 mm in diameter 103. In other embodiments, the main lumen 101 is between about 1.2 and 5 mm in diameter 103. In other embodiments, the main lumen 101 is between about 1.5 and 3 mm in diameter 103. In some embodiments, the main lumen 101 is about 1.83 mm in diameter 103.

In some embodiments, the main lumen 103, or a segment thereof, has a polymeric liner 104 made from a medically acceptable polymer such as, but not limited to, PTFE. In some embodiments, a liner 104 is between about 0.01 and 0.1 mm thick. In other embodiments, a liner 104 is between about 0.02 and 0.05 mm thick. In other embodiments, a liner 104 is about 0.03 mm thick.

In some embodiments, the main lumen 103, or a segment thereof, is encircled by a layer, or multiple stacked layers, of braided wire 105. In some embodiments, the braided wire 104 is outside a liner 104. In some embodiments, the layer(s) of braided wire 105 is between about 0.01 and 0.1 mm thick. In other embodiments, the layer(s) of braided wire 105 is between about 0.03 and 0.07 mm thick. In other embodiments, the layer(s) of braided wire 105 is about 0.05 mm thick.

Still regarding FIG. 2, the inflation lumen 102 is not contiguous with the main lumen 101 and has its distal opening within the balloon 200. In some embodiments, the inflation lumen 102 is embedded within the wall of the catheter 100. Generally speaking, an inflation lumen 102 having a flattened profile allows the overall diameter 109 of the catheter 100 to be as slender as possible. However, the shape of the inflation lumen 102 can be oval, elliptical, circular, square, rectangular, triangular, polygonal or whatever shape profile is suitable for the application of the device. In some embodiments, the inflation lumen 102 has a width 106 of between about 0.1 and about 1 mm. In other embodiments, the inflation lumen 102 has a width 106 of between about 0.2 and about 0.6 mm. In some embodiments, the inflation lumen 102 has a width 106 of about 0.38 mm. In some embodiments, the inflation lumen 102 has a height 107 of between about 0.05 and about 0.3 mm. In other embodiments, the inflation lumen 102 has a height 107 of between about 0.1 and about 0.2 mm. In some embodiments, the inflation lumen 102 has a height 107 of about 0.13 mm.

In some embodiments, the inflation lumen 102 has a polymeric liner 108 made from a medically acceptable polymer such as, but not limited to, polyimide. In some embodiments, a liner 108 is between about 0.01 and 0.1 mm thick. In other embodiments, a liner 108 is between about 0.02 and 0.05 mm thick. In other embodiments, a liner 108 is about 0.03 mm thick.

The overall diameter 109 of the catheter is dependent upon the application for which the occlusion removal device is to be used. For example, a catheter 100 for clearing an obstruction from an intestine will have an overall diameter 109 that is greater than a catheter 100 for clearing an obstruction from an aorta, which in turn will have an overall diameter 109 that is greater than a catheter 100 for clearing an obstruction from a coronary artery or bile duct. In some embodiments, the catheter 100 has an overall diameter 109 of between about 2 and about 20 mm. In other embodiments, the catheter 100 has an overall diameter 109 of between about 2 and about 10 mm. In other embodiments, the catheter 100 has an overall diameter 109 of between about 2 and about 5 mm. In other embodiments, the catheter 100 has an overall diameter 109 of about 2.49 mm.

In some embodiments, a viewing device can be fed through the main lumen 101 for visualization of the occlusion, debris from break-up or reduction of the occlusion, or the cleared bodily lumen. In some embodiments, the viewing device is a nanoscale camera. In some embodiments, a light source can also be fed through the main lumen to illuminate the target site for the viewing device. In some embodiments, the light source is a fiber optic light source.

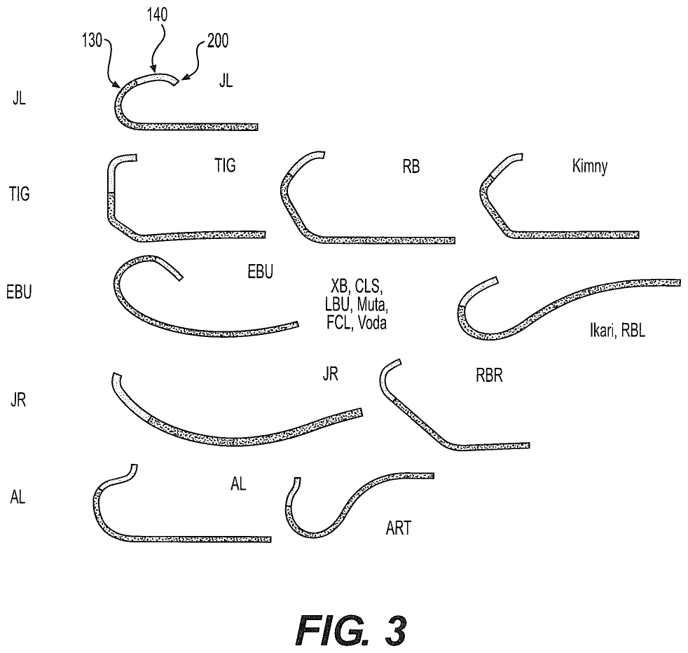

In some embodiments, it can be advantageous to form the catheter 100 to have a distal tip region that is curved. Without wishing to be bound by theory, a curved tip on a catheter 100 may make it easier for the distal tip 160 of the catheter to reach the target site, particularly when the insertion route includes tortuous curves, such as targeting a coronary artery via the aorta, with a femoral, brachial or radial entry point. In general, catheters for femoral, brachial or radial insertion with have different shape profiles at their distal tip. In some embodiments, the catheter 100 has a single curve. In other embodiments, the catheter 100 can have 2, 3, 4, 5 or 6 curves. FIG. 3 illustrates some examples of femoral, brachial or radial specific catheter distal tips. Exemplified here are distal regions having zones of differing stiffness, with the most flexible zone 140 being located most distal and a stiffer zone 130 located proximal thereto. In general, the balloon 200 is located at or near the distal tip of the most distal zone 140. In some embodiments, the most distal zone 140 further comprises a radiopaque marker for tracking the movement of the catheter 100. In some embodiments, the radiopaque marker is distal to the balloon 200. In some embodiments, the radiopaque marker is a tantalum marker.

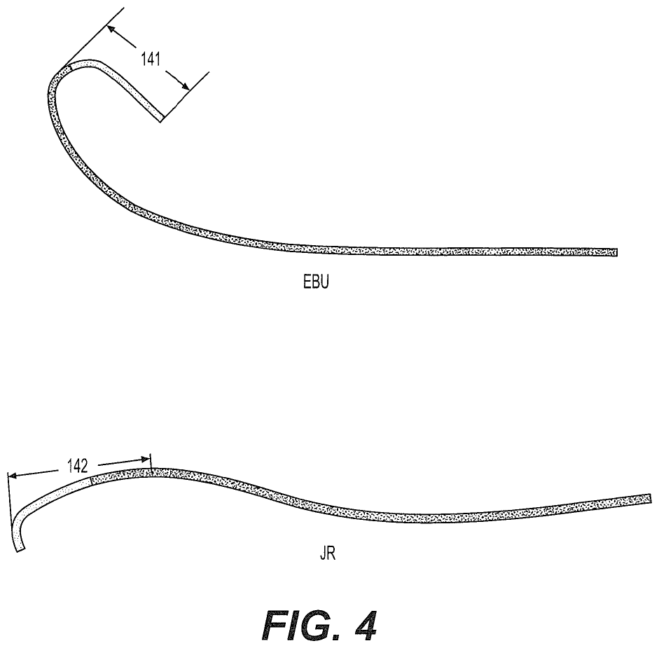

FIG. 4 depicts two types of distal tips for femoral-entry coronary artery catheters, an Extra Backup Curve type (EBU) and a Judkins Right Curve type (JR). In the present examples, the darker region is composed of a stiffer polymer, for example, PEBAX 55D (polyether block amide: Arkema; Columbes, France) and the lighter region is composed of a more flexible polymer, for example, PEBAX 40D (polyether block amide: Arkema; Columbes, France). In the EBU example, the linear distance 141 between the primary curve and the distal tip of the catheter is between about 3 and about 4 cm. In some embodiments, the linear distance 141 between the primary curve and the distal tip of the catheter is about 3.5 cm. In the JR example, the linear distance 142 between the primary curve and the distal tip of the catheter is between about 3.5 and about 4.5 cm. In some embodiments, the linear distance 142 between the primary curve and the distal tip of the catheter is about 4 cm.

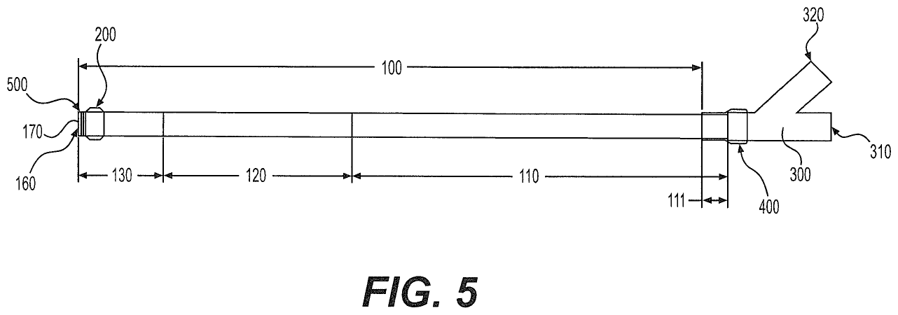

FIG. 5 is a simplified not-to-scale schematic showing an exemplary coronary artery catheter assembly architecture in a catheter having a JR or EBU tip shape. At the proximal end of the assembly is a manifold 300 having a main port 310 and an inflation port 320. The distal end of the manifold 300 is inserted into the proximal end of a strain relief component 400. In some embodiments, about 1 to about 4 cm of the proximal end of the catheter 111 is inserted into the distal end of the strain relief 400. In some embodiments, about 2 to about 3 cm of the proximal end of the catheter 111 is inserted into the distal end of the strain relief 400. In some embodiments, about 2.5 cm of the proximal end of the catheter 111 is inserted into the distal end of the strain relief 400. In some embodiments, the catheter 100 is about 50 to about 150 cm in length. In some embodiments, the catheter 100 is about 75 to about 125 cm in length. In some embodiments, the catheter 100 is about 100 cm in length. A first zone 110 is highest in stiffness. In some embodiments, the first zone 110 is composed of a stiffer polymer, for example, PEBAX 72D (polyether block amide: Arkema; Columbes, France). The second zone 120 is composed of a more flexible polymer, such as PEBAX 55D and is about 10 to about 40 cm in length in some embodiments. In some embodiments, the second zone is about 20 to about 30 cm in length. In some embodiments, the second zone is about 25 cm in length. In some embodiments, both the first zone 110 and the second zone 120 comprise a braided wire sheath as described above in FIG. 2. In the present embodiment, a third zone 130 extends from the distal end of the second zone 120 to the distal tip 160 of the catheter 100. The third zone 130 is the most flexible polymer, such as PEBAX 40D. In some embodiments, the third zone is between about 1.5 and about 5.5 cm in length. In some embodiments, the third zone is between about 2.5 and about 4.5 cm in length. In some embodiments, the third zone is about 3.5 cm in length. In some embodiments, the distal end 160 of the catheter 100 comprises a soft tip 170 that extends beyond the distal end of the balloon 200. In some embodiments, the soft tip has a single lumen that is contiguous with the main lumen 101 of the catheter. In some embodiments, the soft tip 170 extends 1-4 mm beyond the distal end of the balloon 200. In other embodiments, the soft tip 170 extends 2-3 mm beyond the distal end of the balloon 200. In some embodiments, a marker 500 is associated with the soft tip. In further embodiments, the marker 500 is a radiopaque marker. In some embodiments, the radiopaque marker is a tantalum marker. In some embodiments, the marker 500 is located inside the soft tip 170. In some embodiments, the braided wire sheath as described above in FIG. 2 extends to the proximity of the soft tip 170.

In some embodiments, the catheter 100 comprises a wire braid layer as described above in FIG. 2. In some embodiments, the wire braid is composed of a flat braid wire with a thickness between about 0.015 mm and about 0.35 mm. In some embodiments, the flat braid wire has a thickness between about 0.02 mm and about 0.3 mm. In some embodiments, the flat braid wire has a thickness of about 0.025 mm. In some embodiments, the flat braid wire has a width between about 0.1 mm and about 0.16 mm. In some embodiments, the flat braid wire has a width between about 0.115 mm and about 0.145 mm. In some embodiments, the flat braid wire has a width of about 0.13 mm.

In some embodiments, the braid density is between about 10 PPI and 100 PPI. In other embodiments, the braid density is between about 30 PPI and 70 PPI. In still other embodiments, the braid density is between about 40 PPI and 60 PPI. In particular embodiments, the braid density is about 50 PPI. In some embodiments, the braid density is uniform along the length of the catheter 100. In other embodiments, the braid density varies along the length of the catheter 100 dependent upon the needs for a particular region of the catheter 100. In some embodiments, the braid density is higher near the proximal end and lower towards the distal end. In some embodiments, the braid density is about 50 PPI at the proximal end and about 10 PPI at the distal end.

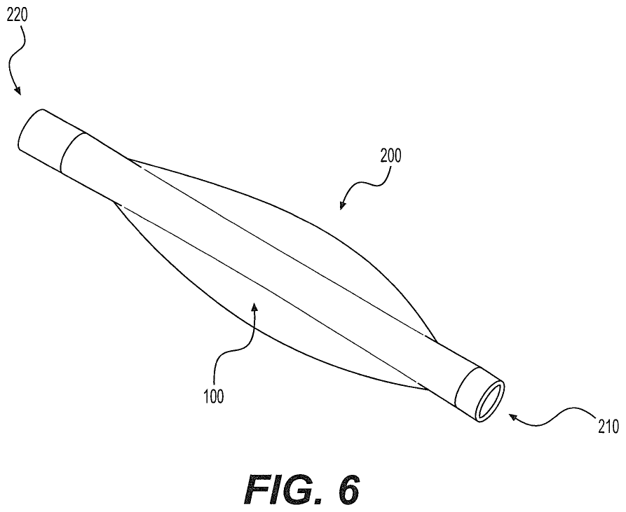

FIG. 6 shows an exemplary balloon 200 of the present occlusion removal device. The balloon comprises a proximal collar 210 and a distal collar 220 for attachment of the balloon to the catheter 100. The distal end of the inflation lumen 102 opens into the space between the balloon 200 membrane and the catheter 100. In some embodiments, the balloon 100 is composed of nylon 12. The balloon 100 must be able to withstand a nominal pressure of 7 ATM. In some embodiments, the balloon 100 must be able to withstand a burst pressure of 10-15 ATM. The balloon material should not contain a total area of embedded particulate material greater than 1.0 sq. mm, per TAPI chart.

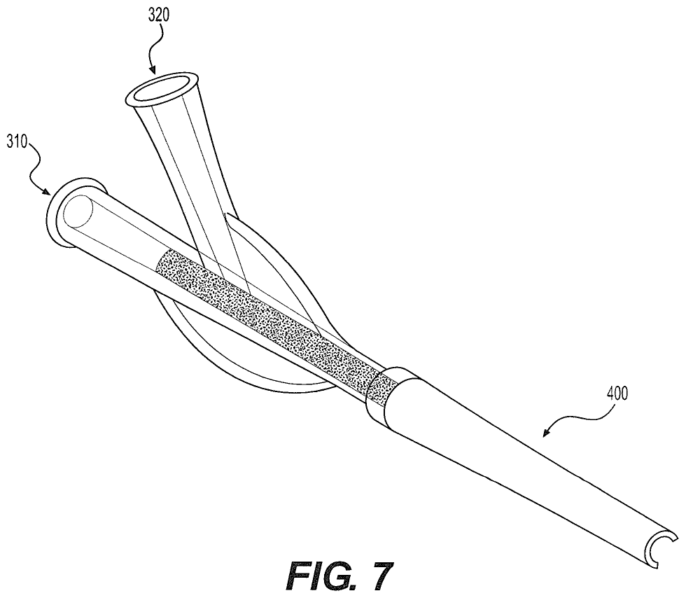

FIG. 7 shows an exemplary manifold 300 and strain relief 400 of the present occlusion removal device. The manifold 300 comprises a main port 310 and an inflation port 320. The strain relief comprises two longitudinal lumens. The first is a main lumen that connects the main port 310 of the manifold 300 to the main lumen 101 of the catheter 100 and allows the passage of an aspiration device or other implements from the main port 310 through the main lumen 101. The second is an inflation lumen that is in fluid communication with the inflation port 320 of the manifold 300 and connects to the inflation lumen 102 of the catheter 100.

### Method for Occlusion Removal

Another aspect of the present application relates to a method for reducing and/or removing an occlusion from a bodily lumen. Examples of bodily lumens treatable by the present method include, but are not limited to arteries, arterioles, veins, venules, nasal passages, sinuses, esophagus, bile duct, pancreatic duct, intestines, ureters and urethra.

The method includes establishing an entry portal at a location that will provide access to the site of the target occlusion. For example, the entry portal can be established by inserting a trocar or Seldinger needle into a blood vessel that provides a path to the site of the target occlusion.

A guide wire is inserted through the entry portal. If a needle was used to establish the portal, the needle is withdrawn from the portal and removed from the guide wire. The guide wire is fed through the entry portal and intervening bodily lumens to near the target site.

The catheter of the aspiration device is threaded over the guide wire and guided to the site of the occlusion. The guide wire is then withdrawn from the catheter.

The balloon on the distal end of the catheter is inflated in order to seal itself against the walls of the lumen, stopping the flow of bodily fluids through the bodily lumen.

An aspiration device is threaded through the main lumen of the catheter and brought into contact with the occlusion. The occlusion is then aspirated out of the bodily lumen. Any type of aspiration device that can be introduced through the main lumen of the catheter is contemplated herein and the nature of the aspiration device may be determined by the location or composition of the instruction. In some embodiments, the aspiration device has a suction hole at the distal end. In other embodiments, the aspiration device has one or more suction holes located on one side or multiple sides of the device in proximity to the distal end.

Clearance of the occlusion from the bodily lumen is confirmed, the balloon is deflated and the catheter is withdrawn through the entry portal.

Optionally, a fiber optic nanoscale camera is advanced through a lumen of the catheter to visually observe the target occlusion or the clearance of the occlusion.

In some embodiments, an obstruction ablation tool is introduced through the main lumen prior to aspiration. In further embodiments, the ablation tool is a rotary ablation tool for breaking up or reducing the occlusion.

In some embodiments, the tool is used to withdraw all or part of the occlusion or occlusion debris through the catheter.

In some embodiments, all or part of the occlusion or occlusion debris is aspirated from the target site through the catheter by applying a suction force through a lumen of the catheter.

Optionally, a fiber optic nanoscale camera is advanced through a lumen of the catheter to visually observe the target site and confirm removal or reduction of the occlusion.

In some embodiments, the balloon is deflated and the catheter is advanced further through the occlusion site to aspirate remaining debris from the break up or reduction of the occlusion.

Following deflation of the balloon, the catheter is withdrawn from the target site and through the entry portal.

### Kit

A third aspect of the present application relates to a kit comprising the aspiration device for occlusion removal as described herein. The kit comprises a multi-lumen catheter having a proximal end and a distal end with an inflatable balloon attached at or near the distal end, and an aspiration device for occlusion break-up, reduction or removal.

In some embodiments, the catheter comprises two longitudinal lumens and attaches at its proximal end to the distal end of a manifold having two ports at its proximal end, wherein each port is in fluid communication with one lumen of the catheter. In some embodiments, a strain relief region is located between the distal end of the manifold and the proximal end of the catheter.

In some embodiments, the kit further comprises an implement for establishing an entry portal. In some further embodiments, the implement is a needle. In some still further embodiments, the needle is a trocar or Seldinger needle.

In some embodiments, the kit further comprises a syringe, which attaches to a port of the manifold, for aspiration of the occlusion through the catheter.

In some embodiments, the kit further comprises a guide wire.

In some embodiments, the kit further comprises a fiber optic viewing device that is extendable through a lumen of the catheter. In some further embodiments, the fiber optic viewing device comprises a nanoscale camera at its distal end.

In some embodiments, the kit further comprises a fiber optic light source that is extendable through a lumen of the catheter.

In some embodiments, the kit comprises two or more aspiration device. The two or more aspiration device can be of the same design, or of different designs.

The invention will be further described with reference to the following non-limiting Example. It will be apparent to those skilled in the art that many changes can be made in the embodiments described without departing from the scope of the present invention. Thus the scope of the present invention should not be limited to the embodiments described in this application, but only by embodiments described by the language of the claims and the equivalents of those embodiments.

### Example 1 Occlusion Removal from Coronary Artery

A middle-aged subject presents at the hospital with myocardial infarction. The subject is determined to have a calcified occlusion of the left anterior descending (LAD) coronary artery.

The groin area of the subject is prepped and a trocar is inserted into the femoral artery between the inguinal ligament and the femoral artery bifurcation.

A J-tipped guide wire is advanced into the femoral artery through the needle and the needle is withdrawn. The guide wire is advanced through the femoral artery, the abdominal aorta, the thoracic aorta and around the aortic arch so that the distal end of the guide wire is in the proximity of the LAD coronary artery.

The distal end of a Judkins left 4 balloon catheter assembly of the present occlusion removal device, having a tantalum marker on its distal end, is advanced talong the guide wire and directed to the opening of the LAD coronary coronary artery under the guidance of fluoroscopy. The guide wire is then withdrawn from the catheter. The catheter is manipulated to place the distal tip of the catheter into the opening of the LAD coronary artery. Contrast solution is injected through the main lumen of the catheter to ensure that the distal tip is in the opening of the vessel.

Normal saline is introduced through the inflation lumen of the catheter to inflate the balloon, temporarily blocking blood flow through the target site.

An aspiration device is advanced through the main lumen of the catheter and contacted with the occlusion. Suction force is applied through the aspiration device, aspirating the debris of the occlusion out of the vessel and through the catheter.

Following clearance of the occlusion, the balloon is deflated and the catheter is withdrawn through the entry portal and the wound is covered.

The above description is for the purpose of teaching the person of ordinary skill in the art how to practice the object of the present application, and it is not intended to detail all those obvious modifications and variations of it which will become apparent to the skilled worker upon reading the description. It is intended, however, that all such obvious modifications and variations be included within the scope of the object of the present application, which is defined by the following claims. The claims are intended to cover the components and steps in any sequence which is effective to meet the objectives there intended, unless the context specifically indicates the contrary.

What is claimed is:

1. A device for occlusion removal comprising a catheter having a proximal end and a distal end, and an aspiration device;
the catheter comprising:
a main lumen having a proximal opening at the proximal end of the catheter and a distal opening at the distal end of the catheter, the main lumen comprising an inner diameter sufficient to accommodate the passage of the aspiration device, and
the aspiration device comprising:
a longitudinal lumen, a connection at the proximal end for a suction device, and at least one opening at or near the distal end.

2. The device for occlusion removal of claim 1, further comprising a manifold at its proximal end, wherein the manifold comprises a main port that is contiguous with the main lumen of the catheter and accommodates the insertion of the aspiration device through the manifold and into the main lumen of the catheter.

3. The device for occlusion removal of claim 2, wherein the main port further attaches to a handle.

4. The device for occlusion removal of claim 1, further comprising a balloon mounted at or near the distal end of the catheter,
the balloon comprising:
a flexible impermeable membrane encircling the catheter, the membrane being bounded by a proximal collar attached to the catheter and a distal collar attached to the catheter; and
the catheter further comprising:
an inflation lumen having a proximal opening at the proximal end of the catheter and a distal opening near the distal end of the catheter, wherein the distal opening is between the proximal and distal collars of the balloon.

5. The device for occlusion removal of claim 4, further comprising a manifold at its proximal end, the manifold comprising:
a main port that is contiguous with the main lumen of the catheter, wherein the main port accommodates the insertion of the aspiration device through the manifold and into the main lumen of the catheter, and
an inflation port that is in fluid communication with the inflation lumen of the catheter, wherein the inflation port is attachable to a syringe or reservoir for inflation of the balloon.

6. The device for occlusion removal of claim 1, further comprising a radio opaque marker at the distal end of the catheter.

7. The device for occlusion removal of claim 6, wherein the radio opaque marker is a tantalum marker.

8. A method for the removal or reduction of an occlusion at a target site in a bodily lumen of a subject in need thereof, comprising the steps of:
a) establishing an entry portal into a site that provides access to the target site,
b) inserting the distal end of the catheter of the device of claim 1 through the entry portal and guiding it to the target site,
c) advancing an aspiration device through the main lumen of the catheter and engaging the occlusion at the target site, and
d) aspirating the occlusion, or fragments thereof, through the catheter.

9. The method of claim 8, further comprising the step of inserting a guide wire through the entry portal prior to step b), wherein the catheter is introduced through the entry portal in step b) by sliding the main lumen of the catheter onto the guidewire.

10. The method of claim 8, wherein engaging the occlusion comprises dislodging it intact for aspiration from the target site.

11. The method of claim 10, wherein engaging the occlusion comprises breaking the occlusion into fragments for aspiration from the target site.

12. The method of claim 8, wherein the bodily lumen is a blood vessel.

13. The method of claim 12, wherein the blood vessel is a coronary artery.

14. The method of claim 12, wherein the occlusion is selected from the group consisting of a thrombus, calcification, lipid and cholesterol.

15. The method of claim 8, wherein the bodily lumen is selected from the group consisting of ureter, urethra, bile duct and pancreatic duct.

16. The method of claim 8, further comprising the step of: e) withdrawing the catheter from the entry portal.

17. A kit, comprising the catheter of claim 1 and at least one aspiration device.

18. The kit of claim 17, further comprising a trocar or Seldinger needle.

19. A device for occlusion removal comprising a catheter having a proximal end and a distal end, a balloon mounted at or near the distal end of the catheter, a manifold having a proximal end and a distal end, and an aspiration device;
wherein the distal end of the manifold attaches to the proximal end of the catheter;
the balloon comprising:
a flexible impermeable membrane encircling the catheter, the membrane being bounded by a proximal collar attached to the catheter and a distal collar attached to the catheter;
the catheter comprising:
a main lumen having a proximal opening at the proximal end of the catheter and a distal opening at the distal end of the catheter, the main lumen comprising an inner diameter sufficient to accommodate the passage of the aspiration device, and
an inflation lumen having a proximal opening at the proximal end of the catheter and a distal opening near the distal end of the catheter, wherein the distal opening is between the proximal and distal collars of the balloon;
the manifold at its proximal end comprising:
a main port that is contiguous with the main lumen of the catheter, wherein the main port accommodates the insertion of the aspiration device through the manifold and into the main lumen of the catheter, and
an inflation port that is in fluid communication with the inflation lumen of the catheter, wherein the inflation port is attachable to a syringe or reservoir for inflation of the balloon; and
the aspiration device comprising:
a longitudinal lumen, a connection at the proximal end for a suction device, and at least one opening at or near the distal end.

20. A method for the removal or reduction of an occlusion at a target site in a bodily lumen of a subject in need thereof, comprising the steps of:
a) establishing an entry portal into a site that provides access to the target site,
b) inserting the distal end of the catheter of the device of claim 19 through the entry portal and guiding it to the target site,
c) inflating the balloon to stop the flow of bodily fluids through the lumen at the target site,
d) advancing an aspiration device through the main lumen of the catheter and engaging the occlusion at the target site, and
e) aspirating the occlusion, or fragments thereof, through the catheter.

21. The method of claim 20, further comprising the step of: f) deflating the balloon.

22. The method of claim 21, further comprising the step of: g) withdrawing the catheter from the entry portal.
